(19) United States
(12) Patent Application Publication
(10) Pub. No.: US 2025/0065030 A1
(43) Pub. Date: Feb. 27, 2025

(54) DISPOSABLE IRRIGATION BASIN FOR EXTREMITY PROCEDURES

(71) Applicant: MDM Wound Ventures, Inc., San Antonio, TX (US)

(72) Inventor: Francis Derk, San Antonio, TX (US)

(21) Appl. No.: 18/946,043

(22) Filed: Nov. 13, 2024

Related U.S. Application Data
(63) Continuation of application No. 16/818,020, filed on Mar. 13, 2020, now abandoned.
(60) Provisional application No. 62/818,436, filed on Mar. 14, 2019.

(51) Int. Cl. A61M 3/02; B65D 1/36; B65D 1/40

(52) U.S. Cl. CPC A61M 3/0287; B65D 1/36; B65D 1/40; B65D 2501/24

(57) ABSTRACT

A disposable basin for extremity procedures, irrigation and lavage in the operating room, treatment clinic or ward allows the extremity to rest comfortably, safely, and securely inside the basin, allowing the surgeon, physician, clinician, and or provider ease of access within a contained protective barrier. Contaminated fluid safely and efficiently drains into an integrated suction trough via the intrinsic design of the basin, resulting in decreased exposure to pathogens, containment of contamination, ease of execution and disposal, and cost savings.

### REFERENCE TO RELATED APPLICATIONS

This application is a continuation of U.S. Nonprovisional patent application Ser. No. 16/818,020, filed Mar. 13, 2020, which claims the priority benefit of U.S. Provisional Patent Application Ser. No. 62/818,436, filed Mar. 14, 2019, the entire content of each of which is incorporated herein by reference.

### FIELD OF THE INVENTION

This invention relates generally to wound cleaning and, in particular, to a disposable irrigation basin for extremity procedures.

### BACKGROUND OF THE INVENTION

Irrigation of extremities utilizes large amounts of sterile saline, water, and cleansing agents. Once in contact with infected and/or bloody extremities, unstable basins can splash or “slosh” contaminated fluid onto and under the sterile field, patient, clinicians, and floors and other surfaces of the treatment venue.

Current extremity irrigation trays are unstable and often times require modification, resulting in poor containment of fluid, contamination, and exposure of blood-borne pathogens. Unstable basins can cost time and money by increasing: (1) cleanup costs (cleaning solutions); (2) time management (room turnover); (3) manpower (housekeeping or Emergency Medical Service (EMS); and (4) expensive resources (towels, chucks, absorbents, etc.) necessary to contain spillage and decontaminate the patient, clinician and treatment area.

### SUMMARY OF THE INVENTION

This invention provides an improved irrigation basin that allows the extremity to comfortably rest in a stabilized position, restricts fluid within, and allows contaminated fluid to safely and efficiently drain into an integrated suction portal. This results in decrease exposure to pathogens and cleanup costs, safe execution, and quick and thorough disposal.

The disposable irrigation basin disclosed herein allows the extremity to rest securely inside the basin and accommodates a tourniquet. An internal extremity resting slope accommodates up to the distal one third of a lower extremity, depending on what level the physician desires. The basin has a built-in step-off groove (underneath) which anchors to the edge of an Operating Room (OR) table, exam chair, exam table, and or any edged examination platform. Once engaged, the distal portion of the basin declines (by design) and is held in place by the body weight of the extremity. As a result, fluids drain into the basin in a “downhill” manner, collect into a series of troughs, and vacate via an integrated suction portal. The contaminated fluid may then travel via suction into Food and Drug Administration (FDA) approved suction canister(s), thus decreasing contamination. The basin can also be used on any flat surface, does not require suction, and can collect up to 1 liter of irrigation/fluid without spillage.

The inventive wound basin may be used before, during, or following any type of extremity intervention, and in any venue. It may be used when the extremity is prepped prior to debridement or intervention, during the procedure to help collect debris, fluid, contamination, or after for irrigation, lavage or rinsing. The extremity is placed gently into the basin with distal part of the extremity lying on the extremity resting surface or slope, with the weight of the extremity keeping the basin in place. As the procedure begins, debris, contaminants, fluids (blood, purulence, drainage) are irrigated away with normal or mechanical irrigation or lavage. If no suction is used, the extremity is still protected from the fluid within the basin since it is elevated above on the resting slope.

### BRIEF DESCRIPTION OF THE DRAWINGS

FIG. 1 is a perspective view of a preferred embodiment of the invention;

FIG. 2 is a top view of the embodiment of FIG. 1;

FIG. 3 is a bottom view of the preferred embodiment;

FIG. 4 is a first side view of the invention;

FIG. 5 is a second side view of the invention;

FIG. 6 is a front view; and

FIG. 7 is a rear view.

### DETAILED DESCRIPTION OF THE PREFERRED EMBODIMENTS

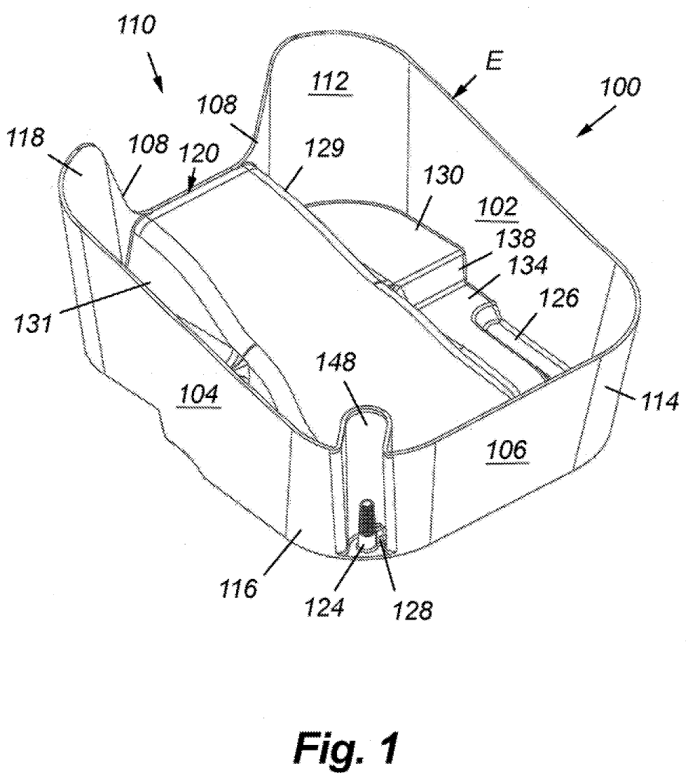

Now making reference to the accompanying drawing, FIG. 1 is a perspective view of a preferred embodiment of the invention, shown generally as basin 100. The basin is preferably constructed as a unitary, molded, single plastic article using a material such as polypropylene. Wall thickness may be generally uniform, with a thickness on the order of ⅛″ to ¼″ to address desired tradeoffs regarding sturdiness, rigidity, weight and cost.

The article is preferably injection molded or thermoformed. It is important to note that while certain dimensions are presented, these are exemplary only and may be varied or adjusted in accordance with a desired application. Indeed, the inventive basis may be provided in different sizes (i.e., adult and child), and mechanisms other than those shown may be used to attach basin to appropriate hospital or treatment room furniture. Materials other than polypropylene may likewise be used.

The basin 100 is generally rectangular in shape, with four sidewalls 102, 104, 106, 108 extending upwardly from a closed bottom to an open top. The sidewalls transition to one another through curved corner sections 112, 114, 116, 118. The front of the basin includes an extremity opening or entry channel 110, with the lower edge 120 of the channel 110 preferably smoothly transitioning to a generally downward sloping ramp 122 forming an extremity resting surface. The preferred embodiment further includes an integrated suction port 124 in fluid communication with the lowest surface internal to the basin, namely, ring-shaped lower trough 126. Fluid communication is facilitated by way of a small passageway from the suction port 124 to the lower surface lower trough 126.

The basin may include additional internal platforms and sloping surfaces forming a series of troughs at different levels. For example, the forward portion of extremity resting surface 122 transitions to first and second side platforms 130, 132 through sloping surfaces 129, 131. Platforms 130, 132 may, in turn, transition to an annular intermediate trough 134 though sloping side surfaces 138, 140. Annular intermediate trough 134 further transitions to lower trough 126 though sloping surface 144. The lower portion of extremity resting surface 122 transitions to trough 134 through sloping surfaces 123, 125.

The purpose of the various internal platforms, sloping surfaces and troughs is two-fold. First, such structures add to overall rigidity. But secondly, and more importantly, the various levels act to keep the work area as clean as possible during procedures. The upper portion of extremity resting surface 122 smoothly transitions to platforms 130, 132. The edge 146 of extremity resting surface 122 smoothly transitions to annular intermediate trough 134, such that the entire lower portion of extremity resting surface 122 transitions to annular intermediate trough 134. With such a construction, fluids and materials readily flow off extremity resting surface 122 to the various levels, ultimately to lower trough 126 and suction port 124, keeping each level relatively free of liquids and debris.

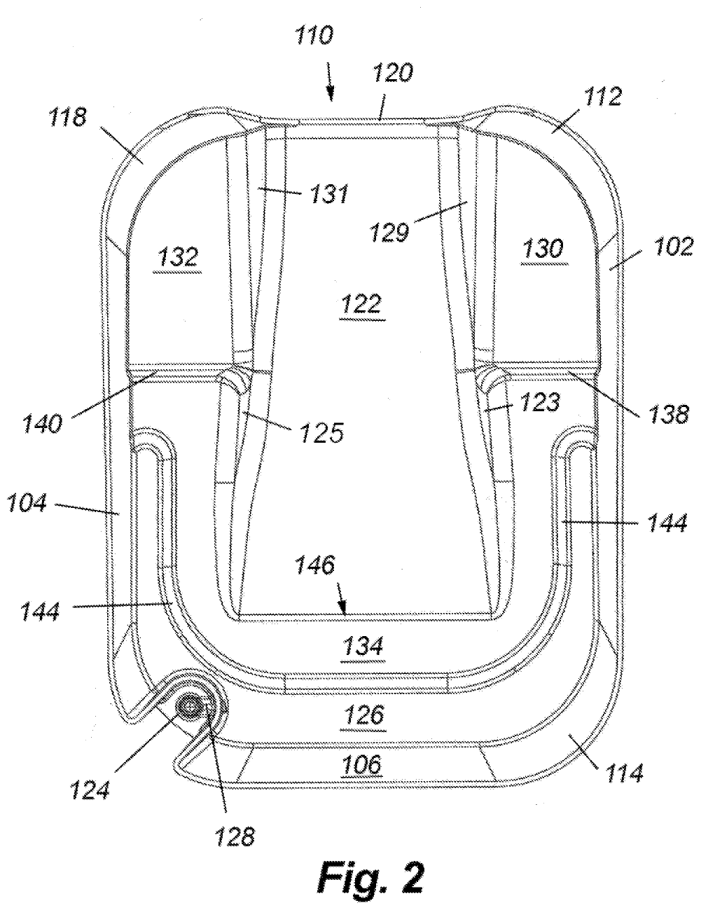

The right and left halves of the basin are generally symmetrical on either side of a central dividing line running from front to back, but for the inclusion of the suction port 124, which is preferably disposed outside the basin proper, surrounded by a semi-circular upwardly extending sidewall 148. FIG. 2 is a top view of the preferred embodiment, perhaps better illustrating all of the various platform levels and sloping surfaces. This figure also better shows passageway 128 from lower trough 126 to suction port 124. Note that the sloping extremity resting surface 122 preferably widens from lower edge 120 to lower edge 146 where the bottom portion of extremity resting surface 122 transitions to intermediate trough 134.

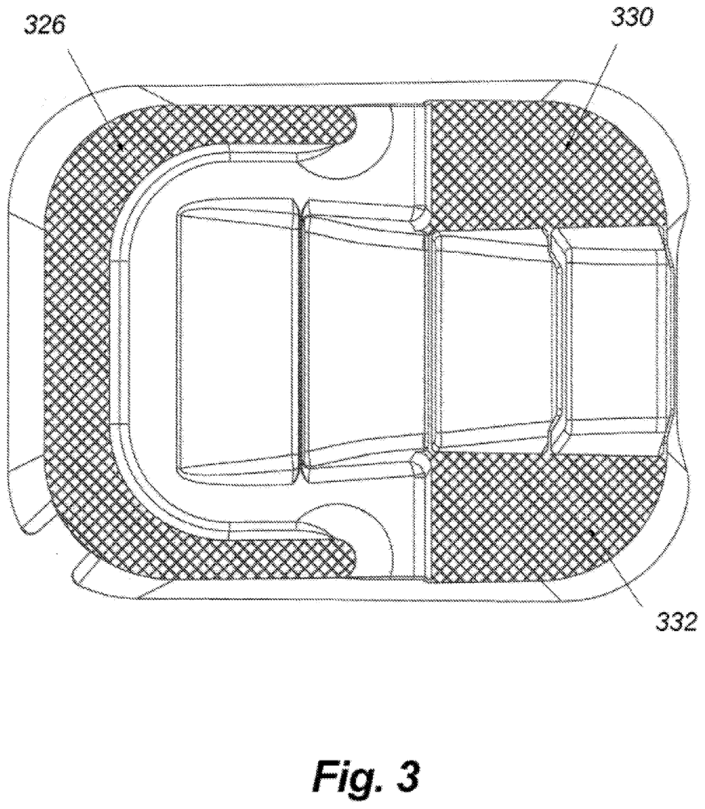

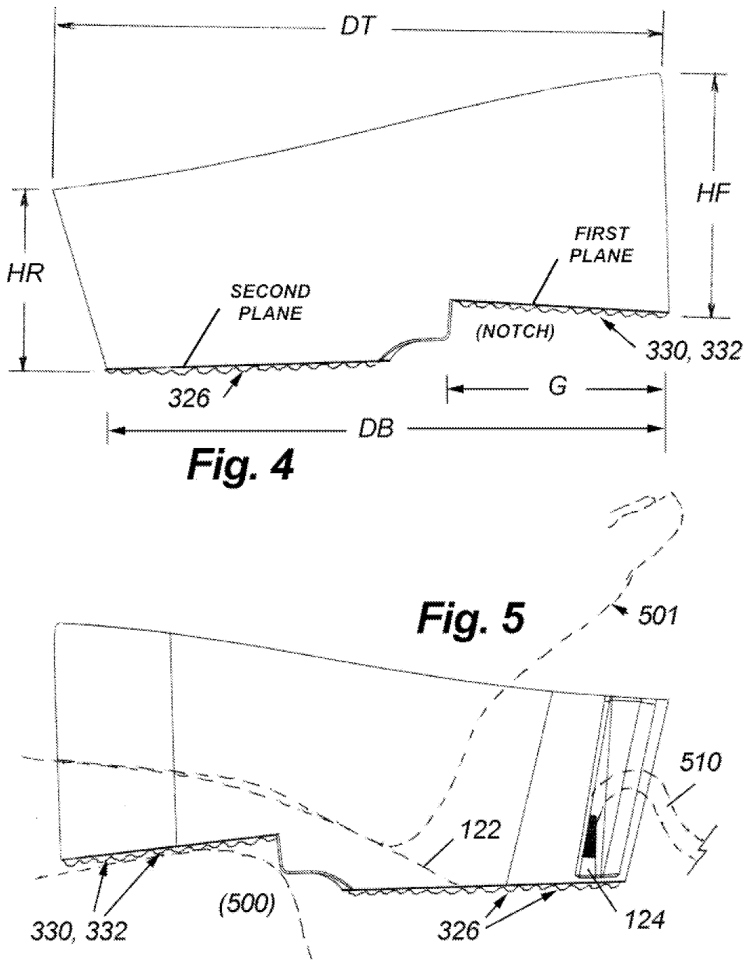

FIG. 3 is a bottom view of the basin showing how particular bottom surfaces preferably include textured surfaces. For example, first and second bottom outer surfaces 330, 332 associated with side platforms 130, 132 are preferably textured, as is the outer surface 326 associated with the underside of lower trough 126. These areas include this texturing to assist in maintaining the basin in position when placed onto a bed or other surface, as discussed with reference to FIGS. 4, 5.

FIG. 4 is a first side view of the invention, and FIG. 5 is a view of the opposing side. In terms of dimensions, the depth of the top edge, DT, is preferably in the range of 11 to 15 inches, more preferably in the range of 12 to 14 inches, and most preferably about 13 inches. The depth of the bottom edge, DB, may be in the range of 10 to 14 inches, more preferably in the range of 11 to 13 inches, and most preferably about 12 inches. Note that overall periphery of the bottom is preferably smaller than the overall periphery of the top edge E to ensure that all of the sidewalls slope downwardly and inwardly toward the bottom.

Continuing the reference to FIGS. 4, 5, the height of the rear wall, HR, may be in the range of 2 to 6 inches, more preferably in the range of 3 to 5 inches, and most preferably about 4 inches. The height of the front wall, HF, may be in the range of 3 to 7 inches, more preferably in the range of 4 to 6 inches, and most preferably about 4 inches. It has been discovered through experimentation, that these numbers and ranges most adequately address adult and child extremities while providing for the most efficient irrigation and cleansing procedures. As mentioned, any or all of these dimensions may be varied depending upon the procedure; for example, the depth of the top edge DT may be made shorter for foot procedures or longer for arm-related operations.

The preferred embodiments further include a step-off groove G that cuts widthwise into the bottom of the basin as shown in FIGS. 4, 5. As shown in FIG. 5, this cut-out anchors the basin to the edge of an OR table, exam chair, exam table, and or any edged examination platform 500. Cross-hatched surfaces 330, 332, 326 enhance stability and grip. Once engaged, the distal portion of the basin declines (by design) and is held in place by the body weight of the extremity 501. As a result, fluids drain into the basin in a “downhill” manner, collect into the series of troughs, to be vacated via integrated suction portal 124. The contaminated fluid then travels via suction through hose 510 into an FDA approved suction canister(s) (not shown), further decreasing contamination. The basin can also be used on any flat surface, does not require suction, and can collect up to 1 liter of irrigation/fluid without spillage.

Prior to this invention, the extremity rests awkwardly on edge or within a simple tray, often causing the distal end of the tray to tip upwards, allowing contaminated fluid to escape. This invention allows the extremity to rest comfortably “inside” the basin on extremity resting surface 122, within the basin and with a stabile weight distribution and direct orientation via an entry channel that is elevated, thereby decreases the chances of splash or “sloshing” of contaminated fluids on the patient, clinician or treatment area.

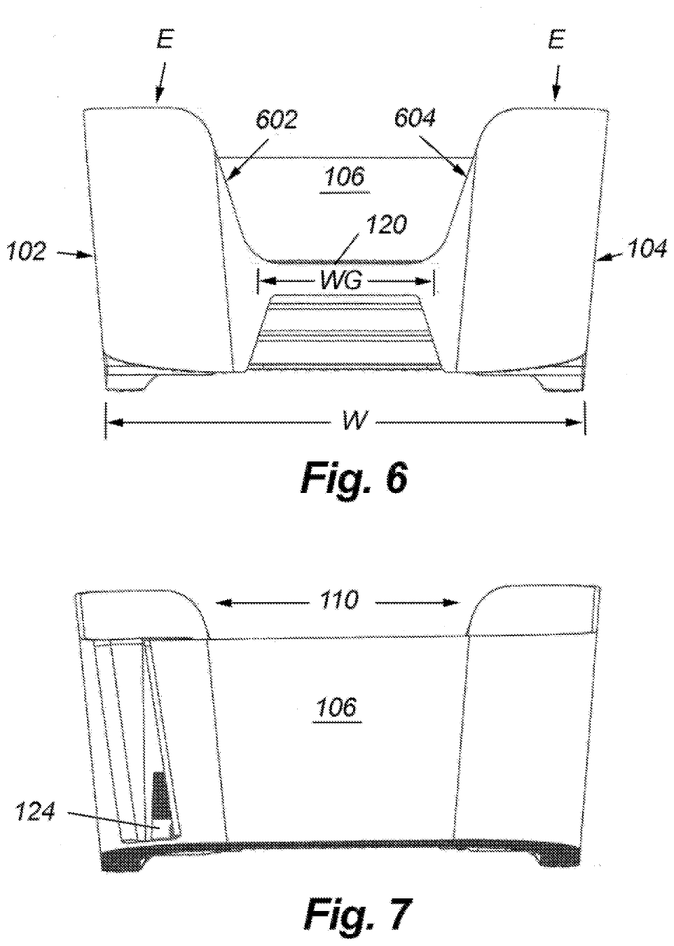

FIG. 6 is a front view of a preferred basin, and FIG. 7 is a rear view. The width W of the lower portion of the basin may be in the range 8 to 12 inches, more preferably in the range of 9 to 11 inches, and most preferably 10 inches, more or less. The width of the upper portion is at least slightly larger to achieve sloping sidewalls 102, 104. The width WG of the lower edge 120 of the extremity opening or entry channel 110 may be in the range of 2 to 5 inches, more preferably in the range of 2.5 to 4.5 inches, and most preferably 3.5 inches, more or less. The sides 602, 604 of the gap preferably slope inwardly and downwardly from the upper edge E, with rounded corners throughout to enhance comfort.

1. An article for irrigating extremities, comprising:
a bottom comprising a suction port and a series of troughs;
a plurality of sidewalls extending from the bottom to define a basin and an open top, wherein at least one sidewall of the plurality of sidewalls defines an entry channel configured to receive an extremity; and
an extremity resting surface extending from the entry channel and into the basin, the extremity resting surface having a first edge opposite a second edge, such that the extremity resting surface slopes downward from the first edge to the second edge, wherein:
the extremity resting surface transitions to a first platform of the series of troughs,
the first platform and the second edge of the extremity resting surface transition to an intermediate platform of the series of troughs,
the intermediate platform transition to a bottom trough of the series of troughs, and
the bottom trough is configured to transport one or both of: materials or fluids to the suction port.

2. The article of claim 1, wherein at least one transition between the extremity resting surface and the first platform, the first platform and the second edge of the extremity resting surface, or the intermediate platform and the bottom trough comprises a sloped surface.

3. The article of claim 1, wherein adjacent sidewalls of the plurality of sidewalls are joined by a curved section.

4. The article of claim 1, wherein at least a portion of a sidewall of the plurality of sidewalls defines a semi-circular sidewall surrounding the suction port.

5. The article of claim 1, wherein the bottom trough is fluidly connected to the suction port through a passageway through a sidewall of the plurality of sidewalls.

6. The article of claim 1, wherein an outer surface of the bottom, underneath the first platform, defines a groove configured to engage with an edged platform and enable drainage of fluids in a downhill manner into the series of troughs.

7. The article of claim 6, wherein at least a portion of the outer surface of the bottom has a textured surface.

8. The article of claim 1, further comprising a hose configured to be fluidly connected to the suction port and a suction canister configured to be fluidly connected to the hose.

9. An article for irrigating extremities, comprising:
a bottom fluidly connected to a suction port;
a plurality of sidewalls extending from the bottom to define a basin and an open top, wherein at least one sidewall of the plurality of sidewalls defines an entry channel configured to receive an extremity;
an extremity resting surface extending from the entry channel and into the basin, the extremity resting surface having a first edge opposite a second edge, wherein the extremity resting surface slopes downward from the first edge to the second edge; and
a plurality of troughs within the bottom, wherein the plurality of troughs have a plurality of levels and a plurality of sloping surfaces such that fluids are configured to flow off the extremity resting surface to the suction port.

10. The article of claim 9, wherein the bottom is fluidly connected to the suction port through a passageway, such that the fluids are configured to flow off of the extremity resting surface through the passageway to the suction port.

11. The article of claim 9, wherein the suction port is separated from the bottom by an upwardly extending sidewall of the plurality of sidewalls.

12. The article of claim 9, wherein the plurality of troughs surround the extremity resting surface.

13. The article of claim 9, wherein the plurality of troughs comprises a first platform, an intermediate platform, and a bottom trough configured to transport one or both of: materials or fluids to the suction port.

14. The article of claim 13, wherein at least one transition between the extremity resting surface and the first platform, the first platform and the second edge of the extremity resting surface, or the intermediate platform and the bottom trough comprises a sloped surface.

15. The article of claim 13, wherein an outer surface of the bottom, underneath the first platform, defines a groove configured to engage with an edged platform and enable drainage of fluids in a downhill manner into the plurality of troughs.

16. The article of claim 15, wherein at least a portion of the outer surface of the bottom has a textured surface.

17. The article of claim 9, wherein adjacent sidewalls of the plurality of sidewalls are joined by a curved section.

18. The article of claim 9, further comprising a hose configured to be fluidly connected to the suction port and a suction canister configured to be fluidly connected to the hose.

19. An article for irrigating extremities, comprising:
a bottom;
a plurality of sidewalls extending from the bottom to define a basin and an open top, wherein at least one sidewall of the plurality of sidewalls defines an entry channel configured to receive an extremity;
an extremity resting surface extending from the entry channel and into the basin, the extremity resting surface having a first edge opposite a second edge, wherein the extremity resting surface slopes downward from the first edge to the second edge; and
a plurality of troughs within the bottom, wherein the plurality of troughs have a plurality of levels and a plurality of sloping surfaces such that fluids are configured to flow off the extremity resting surface to the bottom.

20. The article of claim 19, further comprising a suction port fluidly connected to the basin, wherein the plurality of troughs comprises a first platform, an intermediate platform, and a bottom trough configured to transport one or both of: materials or fluids to the suction port.
